(19) United States
(12) Patent Application Publication
(10) Pub. No.: US 2003/0069272 A1
(43) Pub. Date: Apr. 10, 2003

(54) Method of enhancing joint lubrication with nicotinic acetylcholine receptor agonists

(76) Inventors: Benjamin R. Yerxa, Raleigh, NC (US); Matthew S. Cowlen, Chapel Hill, NC (US)

Correspondence Address: HOWREY SIMON ARNOLD & WHITE, LLP, BOX 34, 301 RAVENSWOOD AVE., MENLO PARK, CA, 94025, US

(21) Appl. No.: 10/268,880

(22) Filed: Oct. 10, 2002

Related U.S. Application Data
(60) Provisional application No. 60/328,571, filed on Oct. 10, 2001.

(51) Int. Cl. A61K 31/4545; A61K 31/4439; A61K 31/44

(52) U.S. Cl. 514/318; 514/343; 514/357

(57) ABSTRACT

The present invention is directed to a method of altering the amount or composition of synovial fluids secreted from joints in a subject in need of such treatment. The method comprises administering to a subject a nicotinic receptor agonist such as nicotine, transmetanicotine, epibatidine, lobeline, and imidacloprid; analogs of such nicotinic agonists; and pyridol and para-alkylthiophenol derivatives in an amount effective to stimulate synovial secretions. Pharmaceutical formulations and methods of their production and administration are also disclosed. The invention is useful for treating disorders associated with joint stiffness, including but not limited to, osteoarthritis and following arthroplastic surgery.

This application claims the benefit of U.S. Provisional Application No. 60/328,571, filed Oct. 10, 2001.

### TECHNICAL FIELD

This invention relates to a method of stimulating the secretion of synovial fluid, mucins, hyaluronic acid, and/or surface active phospholipids, and thereby enhancing joint lubrication, using nicotinic agonists in patients in need of such treatment.

### BACKGROUND OF THE INVENTION

The joint cavity is surrounded by a capsule and held together by ligaments. Synovium lines the joint cavity and is folded upon itself several times to permit considerable motion. The inner portion of the synovium is lined with a layer of synoviocytes, consisting of Type A cells which are involved in phagocytosis and secretion, and Type B cells which are believed to synthesize the hyaluronate of synovial fluid (Bora, et al., Hand Clin. 3: 325-336 (1987)).

Human joints are lubricated by fluid secreted from synovial membranes, which line internal, non-articular joint surfaces. The lubricating properties of synovial fluid have been attributed to a surfactant consisting of surface active phospholipid (SAPL), the mucinous glycoprotein lubricin, hyaluronic acid (hyaluronan), and water (Schwarz, et al., Br. J. Rheumatol. 35: 821-827 (1996), Jay, et al., J. Rheumatol. 27: 594-600 (2000), Marshall, et al., Curr. Opin. Rheumatol. 12: 468-474 (2000), Bora, et al., Hand Clin. 3: 325-336 (1987), Hills, et al., Br. J. Rheumatol. 37: 143-147 (1998), Jay, et al., Connect. Tissue Res. 28: 245-255 (1992), Hills, et al., Proc. Inst. Mech. Eng. 214: 83-94 (2000)). Hyaluronan is a critical constituent component of normal synovial fluid and an important contributor to joint homeostasis. Hyaluronan imparts anti-inflammatory and antinociceptive properties to normal synovial fluid and contributes to joint lubrication, buffering load transmission across articular surfaces and providing a continually replenished source of hyaluronan to articular tissues. (Marshall, Curr. Opin. Rheumatol. 12: 468-474 (2000)).

Joint lubrication is compromised in osteoarthritis (OA) ((Schwarz, et al., Br. J. Rheumatol. 35: 821-827 (1996), Marshall, et al., Curr. Opin. Rheumatol. 12: 468-474 (2000), Hills, et al., Br. J. Rheumatol. 37: 143-147 (1998), Hills, et al., Proc. Inst. Mech. Eng. 214: 83-94 (2000)) and following arthroplastic surgery (Delecrin, et al., Clin. Orthop. 307: 240-249 (1994)). OA is a degenerative joint disease characterized by progressive deterioration and loss of articular cartilage associated with proliferation of new bone and soft tissue in and around the joint. OA may be classified as: (1) primary, in which no underlying cause is apparent; (2) secondary, which is associated with a predisposing factor such as trauma, repetitive stress (occupation, sports), congenital abnormality, metabolic disorder, or other bone/joint disease; and (3) erosive, a syndrome characterized by periods of acute inflammation and progressive destruction of the joints of the fingers, occurring most often in middle-aged women. Unlike rheumatoid arthritis, a systemic disease simultaneously affecting multiple joints, OA involves only joints that are traumatized or exposed to mechanical abuse. OA develops essentially when the rate of wear exceeds the production of new collagen fibers by chondrocytes.

The lipids within the joint, including phospholipid, change in profile shortly after an impact injury leading to eventual OA, whether bone fracture occurs or not (Rabinowitz, et al., Clinical Orthopedics and Related Res. 190: 292-298 (1984)). When SAPL is injected into osteoarthritic joints, wear associated with OA is successfully reduced (Hills, Proc. Inst. Mech. Eng. 214: 83-94 (2000)). In OA, the concentration and molecular weight of hyaluronan in synovial fluid is reduced by dilution, fragmentation, and production by synoviocytes of hyaluronan of lower than normal molecular weight. Consequently, the homeostatic condition of synovial fluid maintained by hyaluronan is compromised (Marshall, Curr. Opin. Rheumatol. 12: 468-474 (2000)). The outermost lubricating layer of SAPL deposited onto articular cartilage from synovial fluid is deficient in OA (Hills and Monds, Br. J. Rheumatol. 37: 143-147 (1998)). Studies of changes in joint fluid after total arthroplasty in a rabbit model of total knee replacement have shown that joint fluid volume and total protein concentration recovers to normal, but hyaluronic acid concentration and molecular weight are reduced and do not completely recover to normal values (Delecrin, et al., Clinical Orthopaedics and Related Research 307: 240-249 (1994)).

The recognition that synovial fluid hyaluronan in OA is abnormal led to the proposition that removal of pathologic osteoarthritic synovial fluid and replacement with products that restore the molecular weight and concentration of hyaluronan toward normal levels can have a beneficial therapeutic effect. The treatment approach has been termed viscosupplementation (Marshall, Curr. Opin. Rheumatol. 12: 468-474 (2000)). Commercial preparations of hyaluronic acid (Healon), which has joint lubricating qualities, have been used as a viscosupplementation treatment for OA (Jay, et al., J. Biomed. Matl. Res. 40:414-418 (1998)).

Therapeutic agents used to manage arthritis include analgesics, anti-inflammatory drugs, muscle relaxants, and antidepressants. Aspirin is the drug of choice for both anti-inflammatory and analgesic reasons. Other non-steroidal anti-inflammatory drugs may be used and act by inhibiting lipo-oxygenase conversion of cell membrane lipids to arachidonic acid. Topical capsaicin cream may help to relieve hand or knee pain and acts by causing the release of the peptide substance P from sensory neurons. Muscle relaxants are used usually in low doses and include diazepam, cyclobenzaprine, carisoprodol, and methocarbamol. Although corticosteroids are not administered orally, they may be administered intra-articularly to reduce inflammation and on an intermittent basis to avoid acceleration of cartilage breakdown. However, the crystalline preparations of corticosteroids may cause synovitis. Purely analgesic agents and tricyclic antidepressants for depression may also be useful. However, nonsteroidal anti-inflammatory drugs (NSAIDS) such as ibuprofen and indomethacin, and newer NSAIDs with specificity for cyclooxygenase-2 (COX-2 inhibitors), including celecoxib, are known to induce gastrointestinal toxicity (Mundasad, et al., J. Ocul. Pharmacol. Ther. 17(2): 173-9 (2001)). Commercial preparations of hyaluronic acid possess inferior lubricating qualities compared with synovial mucin (Jay, et al., J. Biomed. Matl. Res. 40:414-418 (1998)).

Nicotinic acetylcholine receptors are ligand-gated ion channels that regulate a wide range of physiological functions in the central nervous system and innervated tissues, including secretory tissues. Nicotinic agonists are known to induce mucinous secretions in the colon (Finnie, et al., Clin. Sci. 91: 359-364 (1996), stomach (Morris, et al., J. Clin. Gastroenterol. 27 Suppl. 1: S53-63 (1998), nasal passages (Greiff, et al., Thorax 48: 651-655 (1993)), and lung (Peatfield, et al., Clin. Sci. 71: 179-187 (1986)) the latter of which appears to involve the autonomic ganglia that innervate the airway submucosal glands. Nicotine-stimulated fluid secretion is thought to impart a cytoprotective effect on mucosal surfaces of the gastrointestinal tract stomach (Morris, et al., J. Clin. Gastroenterol. 27 Suppl. 1: S53-63 (1998)). Accordingly, nicotinic receptor agonists are being used in the treatment of ulcerative colitis (Guslandi, et al., Br. J. Pharmacol. 48: 481-484, Guslandi, et al., Int. J. Colorectal Dis. 14: 261-262 (1999)). Nicotinic agonists are also being considered as potential therapeutic agents for the treatment of various neurological disorders, including Alzheimer's disease, Parkinson's disease, epilepsy, schizophrenia, and attention deficit hyperactivity disorder (pp. 254, 272, 404, in Neuronal Nicotinic Receptors Pharmacology and Therapeutic Opportunities, Arneric, S. P. and Brioni, J. D. (Eds.), Wiley-Liss, New York (1999); Schnitt and Bencherif, Ann. Rep. Med. Chem. 35: 41-51 (2000)). In addition, nicotine possesses immunosuppressive, anti-inflammatory, and anti-nociceptive (analgesic) properties (pp. 254, 272, 404, in Neuronal Nicotinic Receptors Pharmacology and Therapeutic Opportunities, Arneric, S. P. and Brioni, J. D. (Eds.), Wiley-Liss, New York (1999); Schnitt and Bencherif, Ann. Rep. Med. Chem. 35: 41-51 (2000)). In general, the recent movement to develop novel therapeutics based on nicotinic cholinergic pharmacology has resulted from the identification of distinct nicotinic receptor subtypes and the development of new subtype-selective ligands which can be used to maximize therapeutic effects while minimizing undesirable side effects typically associated with nicotine. Nicotinic agonists have been proposed for therapeutic use as anti-inflammatory and analgesic agents (U.S. Pat. Nos. 3,689,653 and 6,117,889). U.S. Pat. No. 6,277,855 discloses a method for increasing hydration and lubrication of lacrimal tissues using a nicotinic acetylcholine receptor agonist, and is useful for treating dry eye disease and corneal injury. These and all other U.S. patents cited herein are incorporated herein in their entirety.

Acetylcholinesterase inhibitors have been proposed for therapeutic use in the treatment of arthritis, and act by increasing the concentration of the endogenous ligand at the nicotinic receptor, thereby prolonging analgesics and anti-inflammatory effects (International Patent No. WO9729750).

As described above, agents commonly used to treat OA may cause adverse side effects, such as the gastrointestinal toxicity. There exists a need for agents that are both safe and effective in treating OA. The present invention discloses a novel method of enhancing joint lubrication by administering nicotinic receptor agonists.

### SUMMARY OF THE INVENTION

The present invention is directed to a method of altering the amount or composition of synovial fluids secreted from joints in a subject in need of such treatment. The method comprises administering to a subject a pharmaceutical composition comprising a nicotinic acetylcholine receptor agonist in an amount effective to alter the amount or composition of synovial fluids. The nicotinic acetylcholine receptor agonist (nicotinic receptor agonist) is administered in an amount effective to stimulate secretion of synovial fluid, lubricin, hyaluronic acid, or surface-active phospholipids; to enhance joint lubrication, or to treat osteoarthritis. The pharmaceutical compositions useful in the present invention comprise a nicotinic acetylcholine receptor agonist or a combination of the agonist together with a pharmaceutically acceptable carrier therefor.

Nicotinic receptor agonists include but are not limited to: nicotine and its analogs, trans-metanicotine and its analogs, epibatidine and its analogs, pyridol derivatives, piperidine alkaloids such as lobeline and its analogs, certain para-alkylthiophenol derivatives, and imidacloprid and its analogs. The compounds of the present invention are potent agonists of nicotinic receptors; thus, they are useful in the treatment of physiological conditions in which joint lubrication is impaired, such as OA and following arthroplastic surgery.

### DETAILED DESCRIPTION OF THE INVENTION

The present invention provides a method of altering the amount or composition of synovial fluids secreted from joints in a subject in need of such treatment. Components, which determine the lubricating properties of synovial fluid and can be altered by systemic or local treatment with nicotinic acetylcholine receptor agonists, include water, mucinous glycoprotein lubricin, hyaluronic acid, and/or surface-active phospholipids.

Increasing the amount of, or changing the component ratio of synovial fluids can enhance lubrication in joints, and improve disorders associated with reduced joint secretion and lubrication, such as osteoarthritis and complications of knee and hip replacement. Nicotinic acetylcholine receptor agonists interact with nicotinic acetylcholine receptors and stimulate mucinous secretions with lubricating and cytoprotective properties. The method comprises administering to a subject in need thereof a formulation comprising a nicotinic acetylcholine receptor agonist or a combination of nicotinic acetylcholine receptor agonists in an amount effective to alter the amount or composition of synovial fluids from joints such as knee, hip and shoulder.

One embodiment of the present invention is to enhance the secretion of synovial fluid, lubricin, hyaluronic acid, and/or surface-active phospholipids. Another embodiment of the present invention is to increase lubrication in joints. Such method provides for the prevention, management and/or treatment of deficiencies of joint secretion and/or lubrication arising from, but not limited to, arthritis, osteoarthritis, joint surgery, knee and hip replacement, and arthroplastic surgery (joint replacement) in mammals, preferably humans.

The methods of the present invention may be used exclusive of, or as an adjunct to, anti-inflammatory agents, analgesic agents, muscle relaxants, anti-depressants, or agents that promote joint lubrication commonly used to treat disorders associated with joint stiffness, such as arthritis. A combined therapeutic approach is beneficial in reducing side effects associated with agents, such as non-steroidal, anti-inflammatory drugs (NSAIDs), commonly used to prevent, manage, or treat disorders such as OA associated with reduced joint lubrication. In addition to enhancing safety, a combined therapeutic approach is also advantageous in increasing efficacy of treatment.

### DESCRIPTION OF COMPOUNDS

The pharmaceutical compositions useful in this invention comprise a nicotinic acetylcholine receptor agonist (Formula I-X) together with a pharmaceutically acceptable carrier therefor. Useful compositions also include a nicotinic receptor agonist bound to a polymer such as polyethyleneglycol; such compositions are not absorbed systemically. Various nicotine cholinergic receptor agonists are described in Benowitz, et al., P 213-234; Villemagne, et al., p. 235-250; and Holladay, et al., P. 253-270 in Neuronal Nicotinic Receptors, Eds. Arneric and Brioni, Wiley-Liss, Inc. (1999); Vernier, et al., J. Med. Chem. 42: 1684-1686 (1999), and Latli, et al., J. Med. Chem. 42: 2227-2234 (1999). Nicotinic receptor agonists include but are not limited to: nicotine and its analogs, trans-metanicotine and its analogs, epibatidine and its analogs, pyridol derivatives, piperidine alkaloids such as lobeline and its analogs, and certain para-alkylthiophenols.

Nicotinic agonists are depicted by formulae I through X:

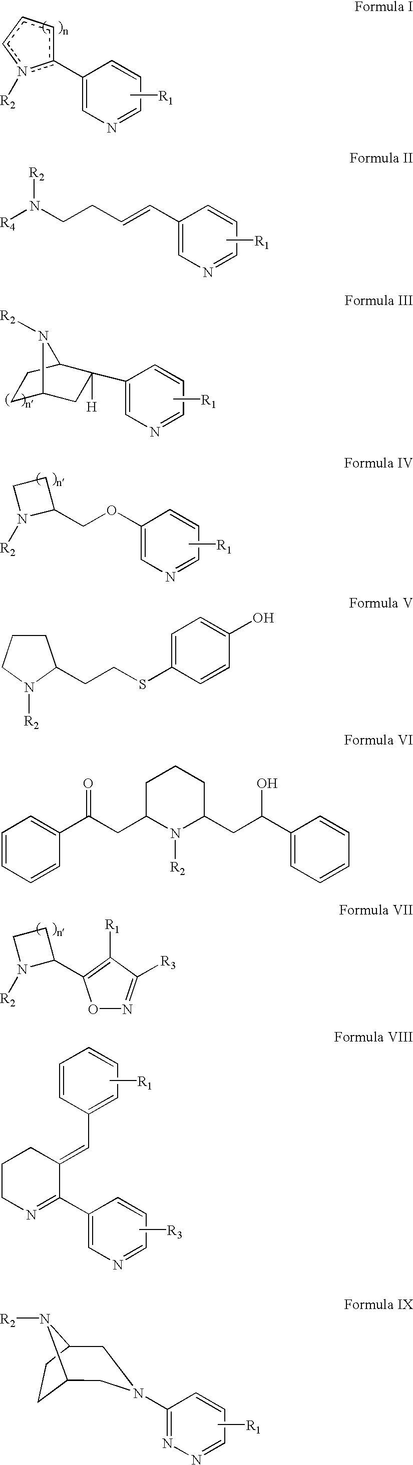

wherein:

n is an integer between 0-3;

n′ is an integer between 1-3;

R₁, and R₃ are H, C₁-C₆ alkyl, C₂-C₆ alkenyl, C₂-C₆ alkynyl, C₃-C₇ cycloalkyl, C₄-C₇ cycloalkenyl, C₁-C₆ alkoxy, F, Cl, Br, I, or amino; wherein at least one hydrogen of said alkyl, alkenyl, alkynyl, cycloalkyl, cycloalkenyl, C₁-C₆ alkoxy, is optionally substituted with a moiety selected from the group consisting of halogen, hydroxy, carboxy, cyano, nitro, sulfonamido, sulfonate, phosphate, sulfonic acid, amino, C₁₋₄ alkylamino, and di-C₁₋₄ alkylamino, wherein said alkyl groups are optionally linked to form a heterocycle; and

R₂ and R₄ are H, C₁-C₆ alkyl, C₂-C₆ alkenyl, C₂-C₆ alkynyl, C₃-C₇ cycloalkyl, C₄-C₇ cycloalkenyl, C₁-C₆ alkoxy, or amino; wherein at least one hydrogen of said alkyl, alkenyl, alkynyl, cycloalkyl, cycloalkenyl, C₁-C₆ alkoxy, is optionally substituted with a moiety selected from the group consisting of halogen, hydroxy, carboxy, cyano, nitro, sulfonamido, sulfonate, phosphate, sulfonic acid, amino, C₁₋₄ alkylamino, and di-C₁₋₄ alkylamino, wherein said alkyl groups are optionally linked to form a heterocycle; optionally R₂ and R₄ in Formula II are linked to form a 5 or 6-membered ring.

The stereochemistry of compounds of Formulae I to X useful in this invention can be either levoratatory (S)-isomer, (R)-isomer, or a mixture of R/S isomers (racemic).

Nicotine analogs of Formula I useful in this invention include nicotine, 5-ethynylnicotine, nornicotine, cotinine, nicotyrine, nicotine-N′-oxide, anabasine, anatabine, myosmine, β-nornicotyrine, N′-methylanabasine, N′-methylanatabine, N′-methylmyosmine, and 2,3′-bipyridyl. Preferred compounds, for example, are: (−)-nicotine, anabasine, and 5-ethynylnicotine.

Preferred compounds of Formula II include trans-metanicotine and 3-ethoxy-trans-metanicotine (without N-methyl group).

Preferred epibatidine analogs of Formula III include epibatidine and its derivatives wherein the chlorine (Cl) on the pyridine ring is replaced by F, Br, I, H, or methyl.

Preferred compounds of Formula IV include [2-methyl-3-(2-(S)-pyrrolidinylmethoxy) pyridine dihydrochloride], ABT-089 (n=2, R₁=1-methyl and R₂=H); -(2-azetidinyl-methoxy)-2-chloropyridine, ABT-594 (n=1, R₁=2-chloro and R₂=H).

Preferred compounds of Formula V include thioalkylphenol derivatives with R₁=methyl, trifluoromethyl, or ethyl. An example of a preferred compound is 4-[[2-(1-methyl-2-pyrrolidinyl)ethyl]thio]phenol hydrochloride (SIB-1553A)

Preferred compounds of Formula VI are lobeline analogs with R₁=CH₃ (lobeline) or R₁=ethyl.

Preferred compounds of Formula VII include (S)-3-methyl-5-(1-methyl-2-pyrolidinyl) isoxazole hydrochloride, ABT-418 (n=2, R₁=3-methyl and R₂=CH₃); and n=2, R₁=ethynyl, R₂=CH₃.

Preferred compounds of Formula VIII include R₁=2,4-dimethoxy (known as DMXB); R₁=2,4-diethoxy; or R₁=2,4-dichloro.

Preferred compounds of Formula IX include R₁=6-chloro and R₂=H (DBO-083); and R₁=6-chloro and R₂=methyl.

Preferred compounds of Formula X include imidacloprid (R₁=C₁, R₂=NO₂), desnitro-imidacloprid (R₁=C₁, R₂=H).

Formulae I-X share a substantial structural feature. The key feature of a nicotinic acetylcholine receptor agonist is the steric and electronic combination of at least one aromatic or heteroaromatic ring and at least one N separated from the ring by 1-6 carbons or 1-6 atoms, preferably 3-5 carbons or atoms. Formulae I-X all display this unifying structural feature despite differences in the N-containing portion, the linking atoms or the aromatic portion.

Some compounds of Formulas I-X can be made by methods known to those skilled in the art; some compounds are commercially available, for example from Sigma Chemical Co. (St. Louis, Mo.). Compounds of Formula I and VIII can be made in accordance with known procedures described by Kem et al (U.S. Pat. No. 5,741,802) and McDonald et al (U.S. Pat. No. 5,723,477). Compounds of Formula II can be made in accordance with known procedures described by Caldwell et al (U.S. Pat. No. 5,861,423). Compounds of Formula III can be made in accordance with known procedures described by Bencherif et al (U.S. Pat. No. 5,922,723), Shen et al (U.S. Pat. No. 5,817,679), and Badio et al. (Eur. J. Pharmacol. 321:189-194 (1997)). Compounds of Formula IV can be made in accordance with known procedures described by Nan-Horng et al (WO/9746554A1). Compounds of Formula V can be made in accordance with known procedures described by Vernier et al., J. Med. Chem. 42:1684-6 (1999). Compounds of Formula VI can be made in accordance with known procedures described by Crooks et al (U.S. Pat. No. 5,830,904). Compounds of Formula VII can be made in accordance with known procedures described by Garvey, et al. J. Med. Chem. 37:4455-63 (1994). Formula X can be made in accordance with known procedures described by Latli et al., J. Med. Chem. 42:2227-34 (1999).

The active compounds of the invention may also be present in the form of their pharmaceutically acceptable salts, such as, but not limited to, an acid salt such as acetates, tartrates, chloride, phosphate, sulfates, sulfites, carbonates, bicarbonate and citrates. Pharmaceutically acceptable salts are salts that retain the desired biological activity of the parent compound and do not impart undesired toxicological effects.

Administration of Novel Compounds

The compounds disclosed herein may be administered to the joint of a patient by any suitable means, such as by topical administration, intra-articular injection or systemic administration. Topical administration includes the use of a solution, gel, suspension, cream, or ointment containing the active compound in a physiologically compatible vehicle. Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Alternatively, the active compounds may be administered by a continuous release device. Those skilled in the art of delivery system development can select using conventional criteria. Solutions formulated for administration to the joint are usually referred to as irrigations. These are sterile solutions, prepared in a manner typical of sterile injections that are intended for prepared as a single use sterile solution.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Another method of topical administration is by delivery through the vagina. Pessaries are solid unit-dose forms suitably shaped for insertion into the vagina and may either be composed of a base that melts at body temperature or which dissolves when in contact with mucous secretions. Examples of suitable bases include, but are not limited to, theobroma oil, synthetic fat bases (e.g. Witepsol), polyethylene glycols (macrogols), and glycerol suppository basis. Vaginal tablets are composed of the active ingredient contained within a solid dosage form base which may include, but not be limited to, excipients such as lactose, microcrystalline cellulose, corn starch, magnesium stearate, silicon dioxide, and hydroxypropyl methylcellulose.

Another means of administration of the active compound to the synovial tissues of the subject involve intra-articular injection of the active compound, such that a therapeutically effective amount of the compound reaches the synovial tissues locally.

A further means of administration of the active compounds is systemically via various methods. One such means involve an aerosol suspension of respirable particles comprised of the active compound, which the subject inhales. The active compound is absorbed into the bloodstream via the lungs and contact the synovial tissues in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Other means of systemically administering the active compounds to the synovial tissues of the subject involve administering a liquid/liquid suspension in the form of nasal drops of a liquid formulation, a nasal spray of respirable particles which the subject inhales, or administration of a nebulized liquid to oral or nasopharyngeal airways. Liquid pharmaceutical compositions of the active compound for producing a nasal spray or nasal drops are prepared by combining the active compound with a suitable vehicle, such as sterile pyrogen free water or sterile saline by techniques known to those skilled in the art.

Other means of systemic administration of the active compound involve oral administration, in which pharmaceutical compositions containing compounds of Formulae I-X are in the form of tablets, lozenges, aqueous or oily suspensions, dispersible powders or granules, emulsion, hard or soft capsules, syrups or elixirs or chewable gum. Compositions intended for oral use may be prepared according to any method known to the art; such compositions may contain one or more agents selected from the group consisting of sweetening agents, flavoring agents, coloring agents, and preserving agents in order to provide pharmaceutically elegant and palatable preparations. Tablets may be prepared to contain the active ingredient in admixture with nontoxic pharmaceutically acceptable excipients which are suitable for the manufacture of tablets. These excipients may be, for example, inert diluents, such as calcium carbonate, sodium carbonate, lactose, calcium phosphate, or sodium phosphate; granulating and disintegrating agents, for example, corn starch or alginic acid; binding agents, for example, starch, gelatin, or acacia; and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example, peanut oil, liquid paraffin, or olive oil.

The active compounds may also be delivered to the synovial tissues of a subject through absorption by the skin using transdermal patches or pads. The active compounds are absorbed into the bloodstream through the skin. Plasma concentration of the active compounds can be controlled by using patches containing different concentrations of active compounds.

Additional means of systemic administration of the active compound to the synovial tissues of the subject involve a suppository form of the active compound, such that a therapeutically effective amount of the compound reaches the synovial tissues via systemic absorption and circulation.

Plasma concentrations of active compounds delivered by any means may vary according to compounds, but are generally 0.1-100 ng/mL; preferably, 0.5-50 ng/mL; and more preferably, 5-25 ng/mL. Topical or local doses vary based on site of delivery, but are generally 0.001-10 mg; preferably, 0.01-5 mg; and, more preferably, 0.05-0.5 mg.

The invention is illustrated further by the following example of treatment, which is not to be construed as limiting the scope to the specific procedures described in them.

### EXAMPLE 1

### Effects of Nicotinic Receptor Agonist in Patients with Osteoarthritis

A formulation of a pharmaceutical composition comprising a nicotinic receptor agonist of Formula I-X, or pharmaceutically acceptable salt thereof, together with a pharmaceutically acceptable carriers is prepared as a sterile solution for administration by intra-articular injection or by a continuous release device. Formulations comprising a pharmaceutical composition of a nicotinic receptor agonist are administered to patients to achieve a plasma concentration range of about 0.1-100 ng/mL; preferably, 0.5-50 ng/mL; and more preferably, 5-25 ng/mL.

Patients demonstrating typical clinical manifestations of the disorder and diagnosis are selected on the basis of pattern of joint involvement, radiographic features, laboratory tests, and synovial fluid findings. At baseline and after treatment with nicotinic receptor agonists, the patients undergo examinations including history, physical examinations by specialists, routine laboratory studies, radiographic assessment, and analysis of joint fluid.

Patient History

Typical symptoms of osteoarthritis include use-related pain affecting one or a few joints with less common rest and nocturnal pain, and brief stiffness after rest or in the morning, lasting less than 30 minutes. Other symptoms include loss of joint movement or functional limitation joint instability, deformity and crepitation (‘crackling’).

Physical Examination

Physical examination reveals chronic monarthritis or asymmetric oligo/polyarthritis and firm or “bony” swellings of the joint margins, such as Heberden's or Bouchard's nodes. Patients rarely display synovitis with a cool effusion. On physical examination, crepitance, an audible creaking or crackling of the joints on movement is sometimes detected. Osteoarthritis is also associated with deformity. Patients display restriction of movement, such as the limitation of internal rotation of the hip. Objective neurologic abnormalities are sometimes observed when the spine is involved and affect intervertebral disks, apophyseal joints and paraspinal ligaments.

Laboratory Studies

Routine laboratory work is normal and conducted to rule out other causes of arthritis such gout, and to detect other primary disorders. Erythrocyte Sedimentation Rate (ESR) is normal but is sometimes elevated in patients with synovitis.

Joint Fluid Analysis

Analysis of the joint fluid provides information about the joint fluid characteristics of osteoarthritis. The joint fluid is normally straw-colored with good viscosity and the number of joint fluid white blood cells (WBC) less than 2000/μL. Analysis of the joint fluid is important in ruling out crystal-induced arthritis or infection.

X-Ray Findings

As the disease progresses and over a long-term duration, radiographic findings include joint space narrowing, subchondral bone sclerosis, subchondral cysts, and osteophytes. Erosions differ from that characteristic of rheumatoid and psoriatic arthritis because they occur subchondrally along the central portion of the joint surface.

Criteria for Therapeutic Efficacy

One of the criteria for determining the effectiveness of treatment with nicotinic receptor agonists is the normalization of various joint fluid characteristics, including but not limited to synovial fluid coloration, viscosity and a WBC count of less than 2,000 μL. In patients with synovitis, normalization of ESR is another criteria for determining the effectiveness of treatment. Additional criteria are reduction in joint related pain, and the improvement of joint movement, including a decrease in joint stiffness, less restriction of joint rotation, and/or a reduction in crepitation.

The invention and the manner and process of making and using it are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method of altering the amount or composition of synovial fluids secreted from joints in a subject in need of such treatment comprising:
administering to a subject a pharmaceutical composition comprising a nicotinic acetylcholine receptor agonist in an amount effective to alter the amount or composition of synovial fluids.

2. The method according to claim 1, wherein said nicotinic acetylcholine receptor agonist is administered in an amount effective to affect a response selected from the group consisting of: enhancing joint lubrication, treating osteoarthritis, and stimulating secretions of synovial fluids, lubricin, hyaluronic acid, or surface-active phospholipid.

3. The method according to claim 1, wherein said nicotinic acetylcholine receptor agonist is selected from the group consisting of compounds of Formula I-X and derivatives thereof: 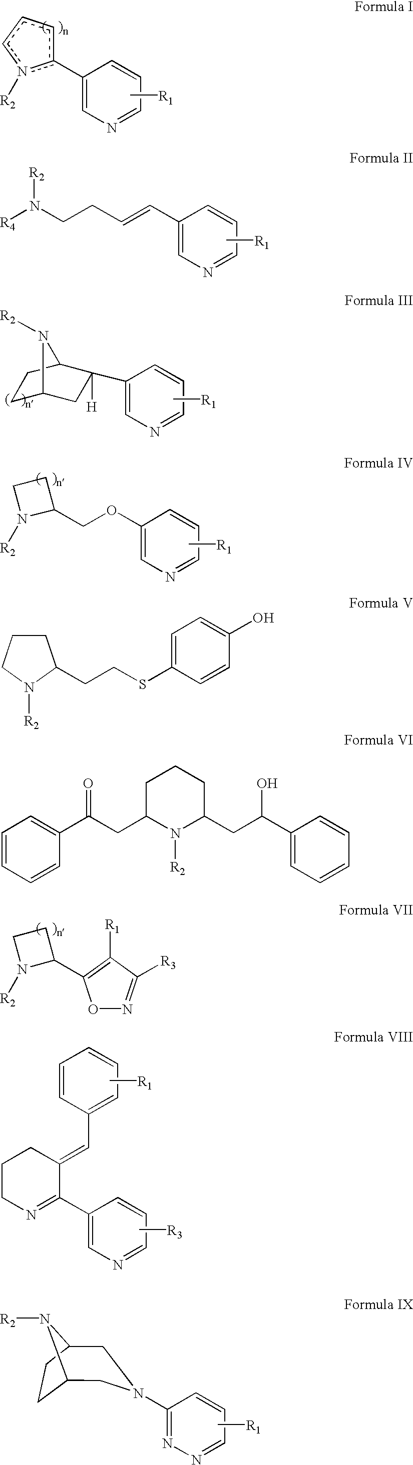
wherein:
n is an integer between 0-3;
n′ is an integer between 1-3;
R₁ and R₃ are H, C₁-C₆ alkyl, C₂-C₆ alkenyl, C₂-C₆ alkynyl, C₃-C₇ cycloalkyl, C₄-C₇ cycloalkenyl, C₁-C₆ alkoxy, F, Cl, Br, I, or amino; wherein at least one hydrogen of said alkyl, alkenyl, alkynyl, cycloalkyl, cycloalkenyl, C₁-C₆ alkoxy, is optionally substituted with a moiety selected from the group consisting of halogen, hydroxy, carboxy, cyano, nitro, sulfonamido, sulfonate, phosphate, sulfonic acid, amino, C₁₋₄ alkylamino, and di-C₁₋₄ alkylamino, wherein said alkyl groups are optionally linked to form a heterocycle; and
R₂ and R₄ are H, C₁-C₆ alkyl, C₂-C₆ alkenyl, C₂-C₆ alkynyl, C₃-C₇ cycloalkyl, C₄-C₇ cycloalkenyl, C₁-C₆ alkoxy, or amino; wherein at least one hydrogen of said alkyl, alkenyl, alkynyl, cycloalkyl, cycloalkenyl, C₁-C₆ alkoxy, is optionally substituted with a moiety selected from the group consisting of halogen, hydroxy, carboxy, cyano, nitro, sulfonamido, sulfonate, phosphate, sulfonic acid, amino, C₁₋₄ alkylamino, and di-C₁₋₄ alkylamino, wherein said alkyl groups are optionally linked to form a heterocycle; optionally R₂ and R₄ in Formula II are linked to form a 5 or 6-membered ring.

4. The method according to claim 3, wherein said nicotinic acetylcholine receptor agonist is nicotine.

5. The method according to claim 3, wherein said nicotinic acetylcholine receptor agonist is trans-metanicotine.

6. The method according to claim 3, wherein said nicotinic acetylcholine receptor agonist is a pyridol derivative.

7. The method according to claim 3, wherein said nicotinic acetylcholine receptor agonist is a piperidine alkaloid.

8. The method according to claim 3, wherein said nicotinic acetylcholine receptor agonist is a para-alkylthiophenol derivative.

9. The method according to claim 1, wherein said pharmaceutical composition is a sterile formulation, which further comprises a pharmaceutically suitable carrier.

10. The method according to claim 1, wherein said pharmaceutical composition is administered to achieve a plasma fluid concentration range of said nicotinic receptor agonist about 0.1 to about 100 ng/mL.

11. The method according to claim 10, wherein said pharmaceutical composition is administered to achieve a plasma fluid concentration range of said nicotinic acetylcholine receptor agonist about 0.5 to about 50 ng/mL.

12. The method according to claim 3, wherein said nicotinic receptor agonist is co-administered with an existing therapeutic agent for managing arthritis.

13. The method according to claim 12, wherein said therapeutic agent is an analgesic agent, anti-inflammatory agent, muscle relaxant, anti-depressant, or agent that promotes joint lubrication.

14. The method according to claim 1, wherein said administering is topical administration of said pharmaceutical composition.

15. The method according to claim 14, wherein said pharmaceutical composition is administered in a form of a solution, a gel, a suspension, a cream, an ointment, a foam, a pessary or a tablet.

16. The method according to claim 1, wherein said administering is systemic or local administration of said pharmaceutical composition.

17. The method according to claim 16, wherein said systemic administration is administered to said subject with said compound in a form selected from the group consisting of: an aerosol suspension of respirable particles; a liquid or liquid suspension for administration as nose drops or nasal spray; a nebulized liquid for administration to oral or nasopharyngeal airways; an oral form; a suppository form; an injectable form; and a transdermal patch or a transdermal pad; such that a therapeutically effective amount of said compound contacts the synovial tissues of said subject via systemic absorption and circulation.

18. The method according to claim 16, wherein said local administration is administered to said subject an injectable form for local intra-articular administration to the affected joint.

19. The method according to claim 17, wherein said oral form is a chewable gum.
